(19) United States
(12) Patent Application Publication
(10) Pub. No.: US 2023/0191128 A1
(43) Pub. Date: Jun. 22, 2023

(54) MODULATION OF FABP4

(71) Applicant: Leonhardt Ventures LLC, Mission Viejo, CA (US)

(72) Inventors: Howard J. Leonhardt, Mission Viejo, CA (US); Jorge Genovese, Irvine, CA (US)

(21) Appl. No.: 18/065,252

(22) Filed: Dec. 13, 2022

Related U.S. Application Data
(60) Provisional application No. 63/291,826, filed on Dec. 20, 2021.
(60) Provisional application No. 63/370,338, filed on Aug. 3, 2022.

(51) Int. Cl. A61N 1/36

(52) U.S. Cl. CPC A61N 1/36034

(57) ABSTRACT

Described is a low voltage, pulsed electrical stimulation device for modulating (e.g., downregulating) expression of fatty-acid-binding protein 4 (“FABP4”) protein(s) by cellular tissues. In certain embodiments, the device is further programmed or alternatively programmed to produce a bioelectric signal or signals that upregulate expression of, for example, klotho by the tissues.

### CROSS-REFERENCE TO RELATED APPLICATION

This application claims the benefit under 35 U.S.C. § 119(e) of U.S. Provisional Patent Application Ser. No. 63/291,826, filed Dec. 20, 2021 and of U.S. Provisional Patent Application Ser. No. 63/370,338, filed Aug. 3, 2022, the disclosures of each of which are hereby incorporated herein in their entirety by this reference.

### TECHNICAL FIELD

The application relates generally to the field of medical devices and associated treatments, and more specifically to precise bioelectrical stimulation of a subject's tissue, possibly augmented with other anti-diabetic therapies. Particularly, the application relates to a device having programmed bioelectric signaling sequences, and associated methods for the controlled modulation of fatty-acid-binding protein 4 (“FABP4”) via precise bioelectrical signaling sequences useful in, for example, treating and/or preventing diabetes. In certain embodiments, the bioelectric signal therapy is supplemented with or substituted by the application of a bioelectric signal or signals that upregulates expression of klotho by the subject's cellular tissue.

### BACKGROUND

Prentice et al. “A hormone complex of FABP4 and nucleoside kinases regulates islet function” Nature (2021) described that uncontrolled or chronic lipolysis associated with insulin resistance and/or insulin insufficiency disrupts metabolic homeostasis. Coupled to this lipolysis is the release of FABP4. Circulating FABP4 levels are strongly associated with cardiometabolic diseases. Prentice et al. showed that hormonal FABP4 forms a functional hormone complex with adenosine kinase (ADK) and nucleoside diphosphate kinase (NDPK) to regulate extracellular ATP and ADP levels. Prentice et al. identified a substantial effect of this hormone on beta cells and given the central role of beta-cell function in both the control of lipolysis and development of diabetes, postulated that hormonal FABP4 is a key regulator of an adipose—beta-cell endocrine axis. Antibody-mediated targeting of this hormone complex improved metabolic outcomes, enhanced beta-cell function, and preserved beta-cell integrity to prevent both type 1 and type 2 diabetes. Thus, the FABP4—ADK—NDPK complex, Fabkin, represents a previously unknown hormone and mechanism of action that integrates energy status with the function of metabolic organs, and represents a promising target against metabolic disease.

Rodriguez-Calvo et al. “Fatty acid binding protein 4 (FABP4) as a potential biomarker reflecting myocardial lipid storage in type 2 diabetes” Clinical Science Vol. 96, pp. 12-21 (2019); DOI: doi.org/10.1016/j.metabol.2019.04.007 concluded that FABP4 was a molecule involved in diabetic/lipid-induced cardiomyopathy and indicated that FABP4 may be an emerging biomarker for diabetic cardiomyopathy-related disturbances, such as myocardial neutral lipid accumulation. Additionally, FABP4 inhibition was identified as a potential therapeutic target for metabolic-related cardiac dysfunctions.

The gene FABP4 is active in adipose tissue and is known to be a contributor to type 2 diabetes. Researchers at Hanyang University in Seoul developed a way to silence the gene using CRISPR interference. Chung et al. “Targeted delivery of CRISPR interference system against Fabp4 to white adipocytes ameliorates obesity, inflammation, hepatic steatosis, and insulin resistance” Genome Res. 2019. 29: 1442-1452. When FABP4 was inhibited in white adipose tissue, lipid storage was reduced. In mice fed a high-fat diet, injecting them twice a week with the CRISPR interference system, the mice lost 20% of their body weight and showed reductions in inflammation and insulin resistance.

### BRIEF SUMMARY

Described is a bioelectric stimulator programmed to produce a bioelectric signal that modulates (e.g., upregulates or downregulates) the expression of FABP4 in a mammalian target tissue.

In certain embodiments, the bioelectric stimulator downregulates the expression of FABP4 in the target tissue. In certain such embodiments, such a bioelectric signal is, within 15%. 20 Hz and a pulse width of 400 μs applied for 30 minutes to 2 hours (e.g., 1 hour) at, for example, 5 mA (as measured at the cellular level of the target tissue).

In certain embodiments, the bioelectric stimulator is further programmed to produce a bioelectric signal that upregulates expression of circulating Klotho, which is useful to treat diabetes and other metabolic disorders, such as atherosclerosis and plaque rupture risk.

A method of using such bioelectric stimulators is to stimulate tissue of a subject, the method comprising: connecting the bioelectric stimulator to the target tissue of the subject, and actuating the bioelectric stimulator to produce the programmed bioelectric signal(s).

The treatment described herein inhibits FABP4 protein production and preferably also increases circulating and local klotho in combination. No other non-pharmacological invention are known to control FABP4 expressions in any form, and pharmacological agents do so very poorly if at all.

In certain embodiments, such a bioelectric signal stimulator is further programmed to produce a bioelectric signal that upregulates expression of klotho. For example, a bioelectric signal of, within 15%, a biphasic current of frequency 20 Hz and a 7.8 ms pulse duration and/or produce at least one bioelectric signal having a frequency selected from the group consisting of 5 Hz, 10 Hz, 20 Hz, 25 Hz, 50 Hz, 75 Hz, 100 Hz, 250 Hz, 500 Hz, 750 Hz, 2,500 Hz, 100,000 Hz, 500,000 Hz, and 1 MHz upregulates expression of klotho in cellular tissue.

Such a bioelectric stimulator may be used to stimulate tissue of a subject, such as by a method comprising: connecting the bioelectric stimulator to the target tissue of the subject, and actuating the bioelectric stimulator to produce the programmed bioelectric signal(s) and modulate expression of the described FABP4 or klotho and/or follistatin.

Typically, in such methods, the subject has been diagnosed with diabetes (e.g., type 2 diabetes mellitus).

In certain embodiments, described is a method of using a bioelectric stimulator to treat a subject for diabetes, the method comprising: connecting the bioelectric stimulator to the target tissue of the subject, e.g., via an electrode, and actuating the bioelectric stimulator to produce programmed bioelectric signal(s), wherein the bioelectric signal(s) downregulate the expression of FABP4 and upregulate expression of klotho.

In certain embodiments, described is a method of treating a subject diagnosed with diabetes (e.g., type 2 diabetes mellitus), the method comprising: connecting a bioelectric stimulator to the target tissue of the subject, wherein the bioelectric stimulator is programmed to produce a bioelectric signal of, within 15%, a biphasic current of frequency 20 Hz and a 7.8 ms pulse duration and/or produce at least one bioelectric signal having a frequency selected from the group consisting of 5 Hz, 10 Hz, 20 Hz, 25 Hz, 50 Hz, 75 Hz, 100 Hz, 250 Hz, 500 Hz, 750 Hz, 2,500 Hz, 100,000 Hz, 500,000 Hz, and 1 MHz, and actuating the bioelectric stimulator to produce the programmed bioelectric signal(s) so as to upregulate expression of klotho in the subject. In such a method, if it is determined that the subject is producing too much klotho, the method can further comprise stopping the initial bioelectric signal, and then actuating the bioelectric stimulator to produce a bioelectric signal having a frequency selected from the group consisting of 25,000 Hz, 50,000 Hz, 750,000 Hz, and 1 MHz.

In certain embodiments, described is a method of treating a subject suffering from cancer utilizing a bioelectric stimulator described herein to downregulate the expression and/or release of FABP4.

Also described is a bioelectric stimulator comprising an electric signal generator and electrodes, which electric signal generator is programmed to produce a bioelectric signal that stimulates target tissue comprising living cells to express and/or release follistatin by the living cells of the target tissue, wherein the bioelectric signal comprises, within 15%, a monophasic positive microcurrent of 100 Hz, 50% duty phase, with a continuous delivery mode for up to five minutes.

As background, serum FABP4 levels are higher in type 2 diabetic patients in comparison to healthy individuals, while klotho levels are lower. FABP4 over-expression interferes with the ability of the patient's pancreas and beta cells to produce healthy insulin levels. At the same time, klotho deficiency causes insulin production to be decreased and insulin sensitivity to be increased.

While FABP4 inhibition attenuates the intracellular lipid content and improves insulin signaling and insulin-stimulated glucose uptake thus reversing effects of type 2 diabetes, Klotho supplementation increases insulin production and improves insulin sensitivity, bringing them back to normal levels.

Other diabetes therapies require injections or infusions of substances to treat diabetes. The described device and methods are non-invasive and control key diabetes reversing protein expression, down regulate FABP4 and up-regulate circulating klotho and/or follistatin, via precise bioelectric signaling sequences applied to key target tissues.

The described bioelectric stimulator and associated methods are useful in treating diabetes and other metabolic disorders including atherosclerosis and plaque rupture risk. Serum FABP4 levels are higher in type 2 diabetic patients in comparison to healthy individuals, while klotho levels are lower. FABP4 over expression interferes with the ability of the pancreas and beta cells to produce healthy insulin levels. Klotho deficiency causes insulin production to be decreased and insulin sensitivity to be increased. FABP4 inhibition attenuates the intracellular lipid content and improves insulin signaling and insulin-stimulated glucose uptake thus reversing effects of type 2 diabetes. Klotho supplementation increases insulin production and improves insulin sensitivity back to normal levels. No other treatment is thought to inhibit FABP4 protein production, while increasing circulating and local klotho. No other non-pharmacological invention controls FABP4 expressions in any form and pharmacological agents do so very poorly if at all. Other diabetes therapies require injections or infusions of substances to treat diabetes. The described device and method are generally non-invasive and control key diabetes reversing protein expression, down regulation of FABP4 and up regulation of circulating klotho, via precise bioelectric signaling sequences applied to key target tissues.

### BRIEF DESCRIPTION OF THE DRAWING

FIG. 1 depicts a programmed bioelectric stimulator for delivery to a subject connected to multiple soft conductive electrode pads.

FIG. 2 depicts a programmed bioelectric stimulator as described herein.

FIG. 3 depicts a conductive soft wrap for use with the system.

### DETAILED DESCRIPTION

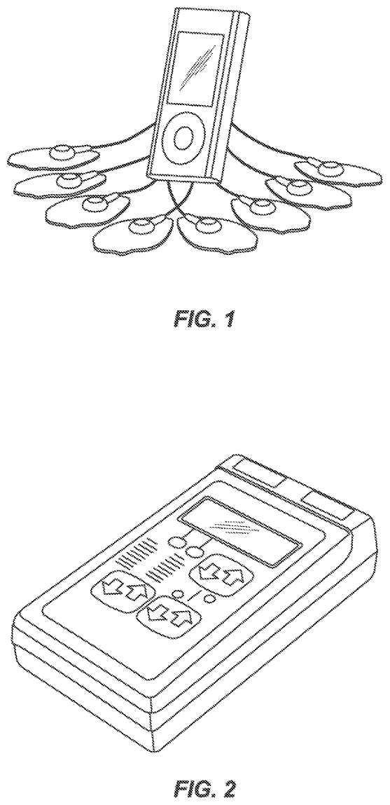

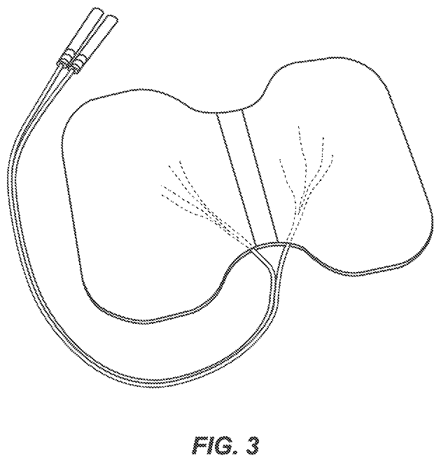

Referring now to FIG. 1, depicted is a biostimulator for use in the treatment of a, for example, human subject.

A micro voltage signal generator for use herein may be produced utilizing the same techniques to produce a standard heart pacemaker well known to a person of ordinary skill in the art. An exemplary microvoltage generator is available from Mettler Electronics Corp. of Anaheim, Calif., US or HTM Electronica of Amparo, BR. The leading pacemaker manufacturers are Medtronic, Boston Scientific Guidant, Abbott St. Jude, BioTronik and Sorin Biomedica.

Construction of the electric signal generators and pacemakers, are known in the art and can be obtained from OEM suppliers as well as their accompanying chargers and programmers. The electric signal generators are programmed to produce specific bioelectric signals to lead to specific protein expressions at precisely the right time for, e.g., optimal treatment or for tissue regeneration.

The biostimulator of FIG. 1 is depicted with multiple soft conductive electrode pads. Electrodes may be used to deliver a bioelectric signal to the subject by applying the electrodes to the subject's skin (e.g., on the skin above the thigh muscles or on the skin above the kidneys).

The biostimulator is actuated and runs through programmed signals to modulate the production of a bioelectric signal or signals that can induce a subject to increase or decrease the expression of, e.g., BMP9 protein for delivery to the subject.

Typical subjects to be treated are mammals such as humans.

Blocking the action of adipokine fatty-acid binding protein 4 (FABP4) through genetic or pharmacological means in a preclinical setting or analyzing the effects of genetically reduced expression in humans has been suggested to protect against obesity-related metabolic derangements and atherosclerosis. Dahlstrom et al. “The low-expression variant of FABP4 is associated with cardiovascular disease in type 1 diabetes” Atherosclerosis, vol. 331, E223-E224, (Aug. 1, 2021).

See, also, Prentice et al. “A hormone complex of FABP4 and nucleoside kinases regulates islet function” Nature 600, 720-726 (2021); doi.org/10.1038/s41586-021-04137-3, showing abnormally high blood levels of fabkin in mice and human patients with either type 1 or type 2 diabetes. Fabkin is composed of a functional protein complex consisting of multiple proteins, including FABP4, adenosine kinase (ADK) and nucleoside diphosphate kinase (NDPK).

In certain embodiments, the bioelectric stimulator is programmed to produce a bioelectric signal of, within 15%, a biphasic current of frequency 20 Hz and a 7.8 ms pulse duration and/or produce at least one bioelectric signal having a frequency selected from the group consisting of 5 Hz, 10 Hz, 20 Hz, 25 Hz, 50 Hz, 75 Hz, 100 Hz, 250 Hz, 500 Hz, 750 Hz, 2,500 Hz, 100,000 Hz, 500,000 Hz, and 1 MHz. As described in U.S. Patent Application Publication US 2020-0289826-A1 to Leonhardt et al. (Sep. 17, 2020) for “Klotho Modulation,” and U.S. patent application Ser. No. 17/473,809 to Leonhardt, filed Sep. 13, 2021, application of such bioelectric signals to a subject's tissue upregulate expression of Klotho.

Utsugi et al. “Decreased insulin production and increased insulin sensitivity in the klotho mutant mouse, a novel animal model for human aging” Metabolism Vol. 49, Issue 9, P1118-1123, Sep. 1, 2000 describes that insulin production is decreased and insulin sensitivity is increased in the klotho mouse.

Typically, by application of the method, the amount of circulating Klotho in the subject's blood stream is increased by at least 20% over normal.

In certain embodiments, klotho expression is upregulated in the subject. Typically, this is done with a bioelectric stimulator programmed to supply at least one bioelectric signal having a frequency of from 5 to 750 Hz, and more typically selected the group consisting of 5 Hz, 10 Hz, 20 Hz, 25 Hz, 50 Hz, 75 Hz, 100 Hz, 250 Hz, 500 Hz, 750 Hz, 2,500 Hz, 100,000 Hz, 500,000 Hz, and 1 MHz. Typically, the bioelectric signal is applied to the subject for from 5 minutes to 24 hours in a day.

In certain embodiments, a bioelectric signal to upregulate the expression and/or release of klotho is a biphasic microcurrent of 20 Hz, 1,000 μs, with a continuous delivery mode for up to thirty minutes per application. In certain applications, the bioelectric signal is applied to the cellular tissue for fifteen (15) minutes.

In certain embodiments, a greater than 25% (e.g., 26.2%) controlled drop in FABp4 levels with the described bioelectric signal is combined with a 150% to 2,300% increase in circulating klotho levels to treat partially reverse type 2 diabetes in a subject. It is known that serum FABp4 levels are higher in type 2 diabetic patients compared to healthy individuals, while klotho levels are lower. FABp4 over-expression interferes with the ability of the pancreas and beta cells to produce healthy insulin levels. Klotho deficiency causes insulin production to be decreased and insulin sensitivity to be increased, thus treating the patient.

Reduced FABP4 gene activity is associated with improved lipid parameters and reduction in cardiometabolic endpoints in man. The small molecules inhibitors of FABP4 are a target for pharmaceutical industry. FABP4 and 5 have an important role in lipid-related metabolic processes and are overexpressed in many cancers (e.g., breast, prostate, colorectal, and ovarian). Moreover, their expression in peritumoral adipose tissue is deregulated, and their circulating levels are upregulated in some tumors.

Higher serum FABP4 has been reported to be useful for the prediction and diagnosis of obesity-related metabolic syndrome and type 2 diabetes mellitus. Lifestyles changes lower FABP4 plasma concentration in patients with cardiovascular risk.

In certain embodiments, klotho expression may be down-regulated (e.g., if it is determined that the klotho has been overexpressed in the patient). Typically, this is done with a bioelectric stimulator programmed to supply at least one bioelectric signal having a frequency selected from the group consisting of 25,000 Hz, 50,000 Hz, 750,000 Hz, and 1 MHz. Typically, the bioelectric signal is applied to the subject for from 5 minutes to 24 hours in a day.

In certain embodiments, the bioelectric stimulator is programmed to produce further bioelectric signals, such as those disclosed in U.S. Pat. No. 10,960,206 to Leonhardt et al. for “Bioelectric Stimulator” (Mar. 20, 2021), the contents of the entirety of which are incorporated herein by this reference. Described therein are bioelectric signals to induce expression by cellular tissue of osteoprotegerin or “OPG”, RANKL, SDF-1, PDGF, a signal for stem cell homing, PDGF, different signals for stem cell proliferation, activin-B, EGF, IGF-1, tropoelastin, VEGF, follistatin, HGF, and any combination thereof.

For example, in the incorporated U.S. Pat. No. 10,960,206, a bioelectric signal to upregulate expression of follistatin in cellular tissue is described (i.e., within 15%, 10 V at 50 Hz and 100 Hz for a duration of, for example, one minute).

In certain embodiments hereof, a bioelectric signal is, for example, utilized to upregulate the expression and/or release of follistatin via a monophasic positive microcurrent of 100 Hz, 50% duty phase, with a continuous delivery mode for up to five minutes per application.

Follistatin is very useful for fighting obesity, osteoarthritis and diabetes. Becker et al. “Follistatin promotes migration and proliferation of glioma cells” European Journal of Cell Biology 89(Supplement 1):17 (March 2010). See also Tang et al. “Gene therapy for follistatin mitigates systemic metabolic inflammation and post-traumatic arthritis in high-fat diet—induced obesity” Science Advances vol. 6, no. 19 (8 May 2020); DOI: 10.1126/sciadv.aaz749.

In certain embodiments, the treatments described herein are further combined with another anti-diabetic therapy or therapies, such as pharmacological treatment, diet, or exercise.

In certain embodiments, the treatments described herein are used in a method of treating a subject suffering from cancer, which treatments utilize a bioelectric stimulator described herein to downregulate the expression and/or release of FABP4. See, e.g., Sun N, Zhao X. “Therapeutic Implications of FABP4 in Cancer: An Emerging Target to Tackle Cancer” Front. Pharmacol. 2022 Jul 11; 13:948610.

### EXAMPLE—Controlling Expression and/or Release of FABP4

Purpose: The purpose of the study is to research the effects of the application of bioelectric signals on FABP4 expression in cells (for example, adipose-mesenchymal derived stem cells (“ADMSCs”)).

Bioelectric Signal: ADMSCs were stimulated using a Mettler microvoltage generator with bioelectric signals at 1 mA, 20 pps, 400 μs for 1 hour and also at 5 mA, 20 pps, 400 μs (as measured at the cellular level) for 1 hour.

Results:

The fold change for the Control was 1.0000.

Although showing overall reduced expression, 1 mA stimulated cells showed no significant difference in the fold change of FABp4 gene expression (p-value: 0.168). Specifically, the fold change was 0.8351 for the ADMSCs treated with the bioelectric signal of 1 mA, 20 Hz, and 400 μs for 1 hour.

5 mA stimulated cells however showed a significant difference in the expression of FABp4 compared to the control (p-value: 0.0099) and reduced gene expression of FABp4. Specifically, the fold change was 0.7380 for the ADMSCs treated with the bioelectric signal of 5 mA, 20 Hz, and 400 μs for 1 hour.

### REFERENCES

(The contents of the entirety of each of which is incorporated herein by this reference.)
Becker et al. “Follistatin promotes migration and proliferation of glioma cells” European Journal of Cell Biology 89(Supplement 1):17 (March 2010).
Chang, Guang-Ping et al. “FABP4 facilitates inflammasome activation to induce the Treg/Th17 imbalance in preeclampsia via forming a positive feedback with IL-17A.” Molecular therapy. Nucleic acids vol. 24 743-754. 2 Apr. 2021, doi:10.1016/j.omtn.2021.03.020.
Chung et al. “Targeted delivery of CRISPR interference system against Fabp4 to white adipocytes ameliorates obesity, inflammation, hepatic steatosis, and insulin resistance” Genome Res. 2019. 29: 1442-1452.
Dahlstrom et al. “The low-expression variant of FABP4 is associated with cardiovascular disease in type 1 diabetes” Atherosclerosis Volume 331, E223-E224, (Aug. 1, 2021).
Furuhashi M and Hotamisligil GS “Fatty acid-binding proteins: role in metabolic diseases and potential as drug targets” Nat Rev Drug Discov. 7(6):489-503 (2008); doi: 10.1038/nrd2589. PMID: 18511927; PMCID: PMC2821027.
Furuhashi, Masato et al. “Fatty Acid-Binding Protein 4 (FABP4): Pathophysiological Insights and Potent Clinical Biomarker of Metabolic and Cardiovascular Diseases.” Clinical Medicine Insights. Cardiology 8 (Suppl 3): 23-33. (2015), doi:10.4137/CMC.S 17067.
Furuhashi, Masato. “Fatty Acid-Binding Protein 4 in Cardiovascular and Metabolic Diseases.” Journal of Atherosclerosis and Thrombosis vol. 26,3 (2019): 216-232. doi:10.5551/jat.48710.
Fuseya, Takahiro et al. “Ectopic Fatty Acid-Binding Protein 4 Expression in the Vascular Endothelium is Involved in Neointima Formation After Vascular Injury.” Journal of the American Heart Association vol. 6,9 e006377. 13 Sep. 2017, doi:10.1161/JAHA.117.006377.
Ge et al. “FABP4 regulates eosinophil recruitment and activation in allergic airway inflammation” Am J Physiol Lung Cell Mol Physiol. 315(2): L227—L240 (2018).
Gharpure et al. “FABP4 as a key determinant of metastatic potential of ovarian cancer” Nat Commun. 2018; 9: 2923.
Li, Guoqing et al. “FABP4 is an independent risk factor for lymph node metastasis and poor prognosis in patients with cervical cancer.” Cancer Cell International vol. 21,1 568. 26 Oct. 2021, doi:10.1186/s12935-021-02273-4.
Liang et al. “Macrophage FABP4 is required for neutrophil recruitment and bacterial clearance in Pseudomonas aeruginosa pneumonia” FASEB J. 33(3):3562-3574 (2019).
Luis et al. “Tumor resistance to ferroptosis driven by Stearoyl-CoA Desaturase-1 (SCD1) in cancer cells and Fatty Acid Biding Protein-4 (FABP4) in tumor microenvironment promote tumor recurrence” Redox Biology vol. 43 (2021): 102006. doi: 10.1016/j.redox.2021.102006.
Miao et al. “FABP4 deactivates NF-κB-IL1α pathway by ubiquitinating ATPB in tumor-associated macrophages and promotes neuroblastoma progression” Clin Transl Med. 2021;11: e395. 10.1002/ctm2.395.
Prentice et al. “A hormone complex of FABP4 and nucleoside kinases regulates islet function” Nature 600, 720-726 (2021); doi.org/10.1038/s41586-021-04137-3.
Rodriguez-Calvo et al. “Fatty acid binding protein 4 (FABP4) as a potential biomarker reflecting myocardial lipid storage in type 2 diabetes” Clinical Science Vol. 96, pp. 12-21 (2019); DOI: doi.org/10.1016/j.metabol.2019.04.007.
Ron et al. “The adipokine FABP4 is a key regulator of neonatal glucose homeostasis” JCI Insight. 2021;6(20):e138288; doi.org/10. 1172/jci. insight. 138288.
Shrestha, Suman et al. “Circulating FABP4 is eliminated by the kidney via glomerular filtration followed by megalin-mediated reabsorption.” Scientific Reports vol. 8,1 16451. 6 Nov. 2018, doi: 10.1038/s41598-018-34902-w.
Sun N, Zhao X. “Therapeutic Implications of FABP4 in Cancer: An Emerging Target to Tackle Cancer” Front. Pharmacol. 2022 Jul 11; 13:948610.
Tang et al. “Gene therapy for follistatin mitigates systemic metabolic inflammation and post-traumatic arthritis in high-fat diet—induced obesity” Science Advances vol. 6, no. 19 (8 May 2020); DOI: 10.1126/sciadv.aaz749.
Tian, Wenying et al. “FABP4 promotes invasion and metastasis of colon cancer by regulating fatty acid transport.” Cancer Cell International vol. 20 512. 19 Oct. 2020, doi: 10.1186/s12935-020-01582-4.
Utsugi et al. “Decreased insulin production and increased insulin sensitivity in the klotho mutant mouse, a novel animal model for human aging” Metabolism Vol. 49, Issue 9, P1118-1123, Sep. 1, 2000; DOI: doi.org/10.1053/meta.2000.8606.
Villeneuve et al. “Unconventional secretion of FABP4 by endosomes and secretory lysosomes” J. Cell Biol. 217(2):649-665 (2018).
Wu, Zimeng et al. “Fatty Acid-Binding Protein 4 (FABP4) Suppresses Proliferation and Migration of Endometrial Cancer Cells via PI3K/Akt Pathway.” OncoTargets and Therapy vol. 14 3929-3942. 29 Jun. 2021, doi:10.2147/OTT.S311792.
Xiao, Yang et al. “Fatty acid binding protein 4 promotes autoimmune diabetes by recruitment and activation of pancreatic islet macrophages.” JCI Insight vol. 6,7 e141814. 8 Apr. 2021, doi:10.1172/jci.insight.141814.
Yan, F et al. “Fatty acid-binding protein FABP4 mechanistically links obesity with aggressive AML by enhancing aberrant DNA methylation in AML cells.” Leukemia vol. 31,6 (2017): 1434-1442. doi:10.1038/1eu.2016.349.
Zhang, Yaqin et al. “High expression of FABP4 and FABP6 in patients with colorectal cancer.” World Journal of Surgical Oncology vol. 17,1 171. 24 Oct. 2019, doi:10.1186/s12957-019-1714-5.
Zhong, Cheng-Qian et al. “FABP4 suppresses proliferation and invasion of hepatocellular carcinoma cells and predicts a poor prognosis for hepatocellular carcinoma.” Cancer Medicine vol. 7,6 (2018): 2629-2640. doi:10.1002/cam4.1511.
U.S. Pat. No. 6,618,625 to Silverstone (Sep. 9, 2003) for “Method and apparatus for treatment of viral diseases”.
U.S. Pat. No. 10,960,206 to Leonhardt et al. (Mar. 30, 2021) for “Bioelectric Stimulator”.
U.S. Patent Application Publication US 2020-0289826-A1 to Leonhardt (Sep. 17, 2020) for “Klotho Modulation”.
U.S. patent application Ser. No. 17/473,809 to Leonhardt, filed Sep. 13, 2021; U.S. Patent Application Publication US 2021/0403184 A1 to Leonhardt (Dec. 30, 2021) for “Klotho Modulation”.
EP 3,262,159 B1 (Jul. 24, 2019) to Gunther et al. for “Genetically Modified Mesenchymal Stem Cell Expressing Klotho”.

What is claimed is:

1. A bioelectric stimulator comprising an electric signal generator and electrodes, which electric signal generator is programmed to produce at least one bioelectric signal that modulates the expression and/or release of fatty-acid-binding protein 4 (FABP4) by a target tissue of a subject comprising living cells.

2. The bioelectric stimulator of claim 1, wherein the bioelectric stimulator downregulates FABP4 in the target tissue.

3. The bioelectric stimulator of claim 1, wherein the at least one bioelectric signal comprises, within 15%, 20 Hz and pulse width of 400 μs.

4. The bioelectric stimulator of claim 2, wherein the at least one bioelectric signal comprises, within 15%, 20 Hz and pulse width of 400 μs.

5. The bioelectric stimulator of claim 1, wherein the bioelectric signal stimulator is further programmed to produce a further bioelectric signal that upregulates expression and/or release of klotho by the target tissue.

6. The bioelectric stimulator of claim 5, wherein the bioelectric signal that upregulates expression and/or release of klotho comprises, within 15%, a biphasic current of frequency 20 Hz and a 7.8 ms pulse duration.

7. The bioelectric stimulator of claim 2, wherein the bioelectric signal stimulator is further programmed to produce a further bioelectric signal that upregulates expression and/or release of follistatin by the target tissue.

8. The bioelectric stimulator of claim 7, wherein the bioelectric signal that upregulates expression and/or release of follistatin comprises, within 15%, a monophasic positive microcurrent of 100 Hz, 50% duty phase, with a continuous delivery mode for up to five minutes.

9. A method of using the bioelectric stimulator of claim 1 to stimulate target tissue comprising living cells of a subject, the method comprising:
connecting the bioelectric stimulator to the target tissue of the subject with the electrodes, and
actuating the bioelectric stimulator to produce the programmed bioelectric signal(s) so as to downregulate the expression of fatty-acid-binding protein 4 (FABP4) by the target tissue.

10. The method according to claim 9, wherein the subject has been diagnosed with diabetes, atherosclerosis, cancer, and/or plaque rupture risk.

11. A method of using the bioelectric stimulator of claim 5 to treat a subject for diabetes, atherosclerosis, cancer, and/or plaque rupture risk, the method comprising:
connecting the bioelectric stimulator to the target tissue of the subject, and
actuating the bioelectric stimulator to produce programmed bioelectric signal(s),
wherein the programmed bioelectric signal(s):
downregulate the expression of fatty-acid-binding protein 4 (FABP4), and
upregulate the expression of klotho.

12. A method of using the bioelectric stimulator of claim 7 to treat a subject for diabetes, atherosclerosis, cancer, and/or plaque rupture risk, the method comprising:
connecting the bioelectric stimulator to the target tissue of the subject, and
actuating the bioelectric stimulator to produce programmed bioelectric signal(s),
wherein the programmed bioelectric signal(s):
downregulate the expression of fatty-acid-binding protein 4 (FABP4), and
upregulate the expression of follistatin.

13. The method according to claim 12, wherein the bioelectric signal that upregulates expression and/or release of follistatin comprises, within 15%, a monophasic positive microcurrent of 100 Hz, 50% duty phase, with a continuous delivery mode for up to five minutes.

14. A bioelectric stimulator comprising an electric signal generator and electrodes, which electric signal generator is programmed to produce a bioelectric signal that stimulates target tissue comprising living cells to express and/or release follistatin by the living cells of the target tissue, wherein the bioelectric signal comprises, within 15%, a monophasic positive microcurrent of 100 Hz, 50% duty phase, with a continuous delivery mode.
